(19) United States
(12) Patent Application Publication
(10) Pub. No.: US 2012/0004655 A1
(43) Pub. Date: Jan. 5, 2012

(54) Bipolar Connector System

(76) Inventors: Harrison Jay Kim, Irvine, CA (US); Sergey Marker, Irvine, CA (US); David C. Leingang, Costa Mesa, CA (US)

(21) Appl. No.: 12/826,894

(22) Filed: Jun. 30, 2010

(51) Int. Cl. A61B 18/14; H01R 29/00

(52) U.S. Cl. 606/41; 439/171

(57) ABSTRACT

An electrical connector assembly comprises a first elongated connector in parallel alignment with a second elongated connector and an adjustment mechanism operable to control movement of the first elongated connector relative to the second elongated connector while maintaining parallel alignment between the elongated connectors.

### TECHNICAL FIELD

This disclosure relates in general to a system for connecting instruments to a console and more particularly to a system for connecting bipolar medical instruments to a console.

### BACKGROUND

Electrosurgical instruments use high frequency electrical energy to cut, coagulate, cauterize, or otherwise treat biological tissue. The instruments, when in contact with tissue, allow for the passage of a high frequency current along a pathway from an active electrode, through tissue, and then to a ground or return electrode. The current flow allows the surgeon to cut or coagulate tissue by varying parameters such as power, contact time, wave form, frequency, etc. Electrosurgical instruments may have various configurations including probes, scissors, and forceps.

There are two common types of electrode configurations used in electrosurgical systems: monopolar and bipolar configurations. In monopolar electrosurgery, current flows from a generator to a single active electrode, such as a lancet or scalpel held by a surgeon. The current passes through the patient's body to a dispersive pad, which is the ground electrode, and back to the generator. The dispersive pad covers a large portion of the patient's body relative to the size of the active electrode, thus preventing tissue damage or significant heat buildup by allowing the current to spread over a larger area.

In bipolar electrosurgery, current flows from the generator to a surgical instrument, such as forceps. One tine of the forceps acts as the active electrode and directs the current through the patient tissue to the other tine, which acts as the return electrode and enables the flow to return to the generator to complete the circuit. A dispersive pad is not required for bipolar surgery.

Typically, electrosurgical instruments are connected to a generator housed in a console using a connecting device such as a connector cable. Cables to connect bipolar instruments to the console typically have two electrical connectors while monopolar instruments only require one connector. The bipolar electrical connectors may be configured as separated “flying leads,” but this configuration may pose a safety concern. For example, one of the connectors may be inserted into the console while the other remains unconnected or the other lead may be inadvertently inserted into the wrong receptacle on the console.

Accordingly, there is a need for improved connector cables, particularly for bipolar cables that can minimize the risk of improperly connecting the cable to the electric generator console. There is also a need for connector cables that may comply with increased safety and utility standards such as those promulgated by the International Electrotechnical Commission (IEC) under the guidelines of IEC 60601-2-2.

### SUMMARY

In one exemplary aspect, an electrical connector assembly comprises a first elongated connector in parallel alignment with a second elongated connector and an adjustment mechanism operable to control movement of the first elongated connector relative to the second elongated connector while maintaining parallel alignment between the elongated connectors.

In another exemplary aspect, an electrosurgical system comprises a bipolar surgical instrument and an electrical connector assembly to connect the bipolar surgical instrument to one or more surgical consoles. The electrical connector assembly includes a first elongated connector in parallel alignment with a second elongated connector and an adjustment mechanism operable to control movement of the first elongated connector relative to the second elongated connector while maintaining parallel alignment between the elongated connectors.

In another exemplary aspect, a method comprises selecting a bipolar surgical instrument and selecting a first surgical console for electrical connection to the bipolar surgical instrument. The first surgical console includes a first set of paired receptacle ports separated by a first spacing. The method further includes selecting a connection assembly. The connection assembly interconnects the bipolar surgical instrument and the first surgical console. It includes a first elongated connector in parallel alignment with a second elongated connector and an adjustment mechanism. The method further includes operating the adjustment mechanism to move the first elongated connector relative to the second elongated connector while maintaining parallel alignment between the elongated connectors until each of the elongated connectors is in alignment with one of the receptacle ports in the first set. Once aligned, each of the elongated connectors is inserted into one of the receptacle ports of the first set to connect the bipolar surgical instrument to the first surgical console.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is a diagram of a bipolar electrosurgical system according to one embodiment of the present disclosure.

FIGS. 2-5 depict various configurations of connector assemblies according to embodiments of the present disclosure.

FIGS. 6-8 are end views of connector assemblies according to embodiments of the present disclosure.

FIG. 9 is another configuration of a connector assembly according to an embodiment of the present disclosure.

FIG. 10 is an end view of the connector assembly of FIG. 9.

FIG. 11 is an end view of a connector assembly according to another embodiment of the present disclosure.

FIG. 12 is a method for using a connector assembly according to an embodiment of the present disclosure.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

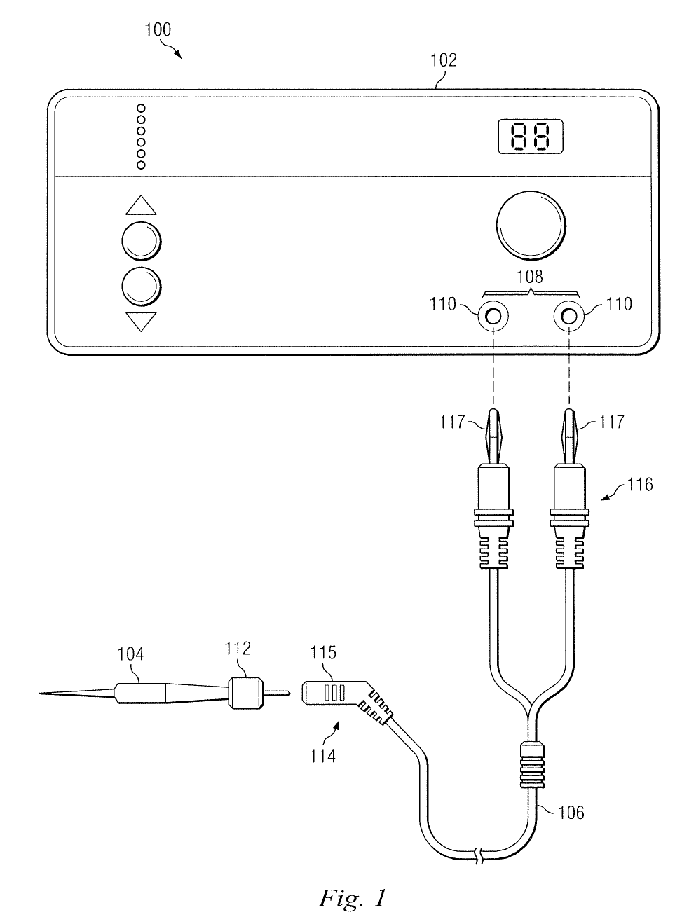

FIG. 1 depicts an electrosurgical system 100 including a console 102, a surgical instrument 104, and a connector assembly 106. The console 102 houses or is connected to an electric generator (not shown). The console includes a receptacle 108 for connecting the connector assembly 106 to the console and thereby to the electric generator. In this example, the receptacle 108 includes two receptacle ports 110 sized and shaped to connect with the bipolar connector assembly 106. In alternative embodiments, the receptacle may include more or fewer receptacle ports.

In this embodiment, the surgical instrument 104 may be a bipolar electrosurgical instrument, such as forceps, scissors, or clamps. The instrument 104 may include active and return electrodes (not shown). The instrument 104 may further include a coupling 112 to connect the instrument with power cabling.

The connector assembly 106 is a power cabling system having a proximal end 114 which includes a coupling 115 sized and shaped to fasten with the coupling 112 of the surgical instrument 104 for removably connecting the instrument and the connector assembly. In this embodiment, the surgical instrument 104 may be disconnected and swapped for a different instrument without disconnecting the connector assembly from the console 102. In alternative embodiments, the instrument 104 may be wired directly to the connector assembly, eliminating the need for the couplings 112, 115.

The connector assembly also has a distal end 116 which includes a pair of connectors 117 sized and shaped to connect with the receptacle ports 110 of the console 102. In this embodiment and others described in this disclosure, connectors are elongated and configured as “banana plugs,” but other suitable connectors, including pin or clips, may be used. The connectors may, in alternative embodiments, be shrouded. The connectors 117 are movable relative to one another in three dimensions and are of a type commonly know as “flying leads.” The freely movable flying leads permit the connector assembly 106 to be used with different consoles having different spacings between receptacle ports 110.

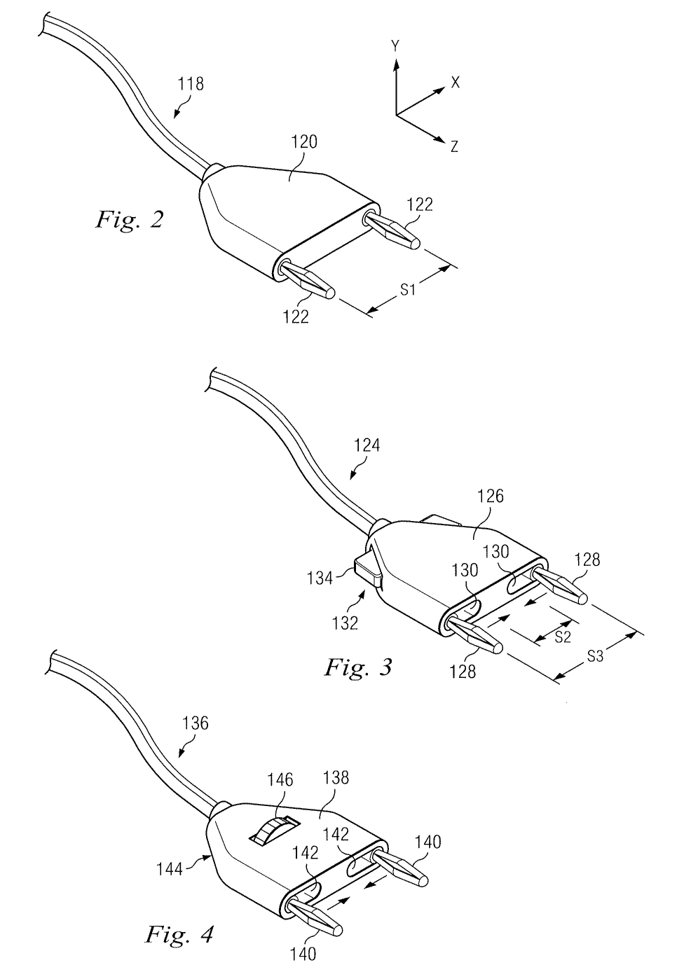

To reduce the likelihood that connector pairs may be improperly connected, the connectors may be fixed relative to one another. FIG. 2 illustrates a portion of a connector assembly 118 including a connector body 120 and a pair of connectors 122. In this embodiment, the connectors 122 are in generally parallel alignment and fixed relative to each other in three dimensions X,Y, and Z. The parallel spacing between the connectors 122, in this embodiment is identified as S1. This configuration of connectors may minimize the likelihood that one connector will be inserted into a console receptacle port while the other connector remains unconnected or becomes connected to another opening in the console. This configuration, however, may limit the use of the connector assembly to consoles having a spacing S1 between the receptacle ports. Consequently, the user may need to purchase and have readily available many different connector assemblies to connect different instruments to different consoles.

To minimize the need for multiple connector assemblies, the spacing between the connectors may be adjusted in a controlled manner. For example, FIG. 3 illustrates a connector assembly 124 including a connector body 126 and a pair of connectors 128 in parallel alignment. The connector body 126 includes a pair of slots 130 through which the connectors 128 are connected to the connector body 126. The slots 130 permit linear translation of the connectors 128 along the X axis, maintaining the connectors in parallel alignment. Thus, the connectors 128 may be variably positioned relative to one another along the X axis, but fixed relative to one another along the Y and Z axes. The connector assembly 124 includes an adjustment mechanism 132 which includes an activating assembly 134 and a spring assembly (not shown) housed within the connector body 126. The activating assembly 134 may include a depressible or squeezable controller or a pair of controllers located on opposite, ergonomically located sites on the connector body. Squeezing the controllers, for example, may activate the spring assembly to move the connectors 128 together or apart, in parallel alignment, along the X axis. For example, the activating assembly 134 may operate to move the connectors 128 within the slots 130 from a spacing S2 to a spacing S3. This adjustability may allow the connectors 128 to fit a variety of consoles having different receptacle port spacings.

FIG. 4 illustrates a connector assembly 136 including a connector body 138 and a pair of connectors 140. The connector body 138 includes a pair of slots 142 through which the connectors 140 are connected to the connector body 138. The slots 142 permit linear translation of the connectors 140 along the X axis. In this embodiment, the connector assembly 136 includes an adjustment mechanism 144 which includes an activating assembly 146 and a gear assembly (not shown) housed within the connector body 138. The activating assembly 146 may include a thumbwheel operational within the connector body 138. Rotating the thumbwheel, for example, may activate the internal gear assembly to move the connectors 140 together or apart, in parallel alignment, along the X axis to adjust the spacing between the connectors. This adjustability may allow the connectors 140 to fit a variety of consoles having different receptacle port spacings.

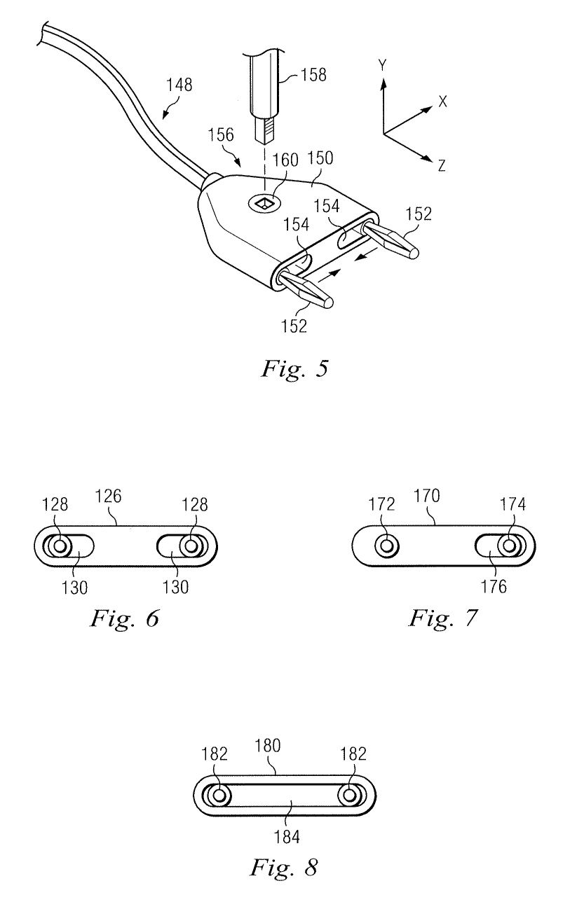

FIG. 5 illustrates a connector assembly 148 including a connector body 150 and a pair of connectors 152. The connector body 150 includes a pair of slots 154 through which the connectors 152 are connected to the connector body 150. The slots 154 permit linear translation of the connectors 152 along the X axis. In this embodiment, the connector assembly 148 includes an adjustment mechanism 156 which includes a tool 158, a tool engagement mechanism 160 and an internal movement assembly (not shown) housed within the connector body 150. The tool 158 may be, for example, a screwdriver or pliers capable of mating with, attaching to, grabbing, or otherwise connecting with the tool engagement mechanism 160. Moving the tool engagement mechanism 160 may activate the internal movement assembly to control movement of the connectors 152 together or apart, in parallel alignment, along the X axis to adjust the spacing between the connectors. This adjustability may allow the connectors 152 to fit a variety of consoles having different receptacle port spacings.

In addition to the adjustment mechanisms described above, other types of adjustment mechanisms including slide controls, ratchet systems, electrical, electromechanical, magnetic, or other types of adjustment mechanisms may be used to control movement of the elongated connectors relative to one another while maintaining parallel alignment. As described, the adjustment mechanisms may provide continuous adjustability, however in alternative embodiments, the adjustment mechanism may include a switch, dial, or other mechanism to permit the spacing between the connectors to be changed in discrete increments. As explained, the adjustment mechanisms described in this disclosure may be configured to allow the connectors to fit a variety of consoles having different receptacle port spacing. For example, the spacing between connectors may be adjustable between 0.50 and 1.50 inches, and adjustability between 0.75 and 1.25 inches may be particularly desirable. These spacings are merely examples, and one skilled in the art would understand that the adjustment mechanism may be configured to provide greater or reduced connector spacing.

FIG. 6 is an end view of any of the connector body 126 of FIG. 3 with the two elongated slots 130 through which the connectors 128 protrude. FIG. 7 depicts an alternative connector body 170 with one fixed connector 172 and one adjustable connector 174. The adjustable connector 174 is movable using any of the adjustment mechanisms described in this disclosure within slot 176. Any of the adjustment mechanisms described above may be used to vary the location of the connector 174 within the slot 176. FIG. 8 depicts another alternative connector body 180 with connectors 182 adjustable using any of the adjustment mechanisms previously described. In this embodiment, both connectors 182 are movable within an opening 184 in the connector body 180. In alternative embodiments, the slots or openings in the connector bodies may be widened or oriented to permit movement of the connectors in other directions on an XY plane.

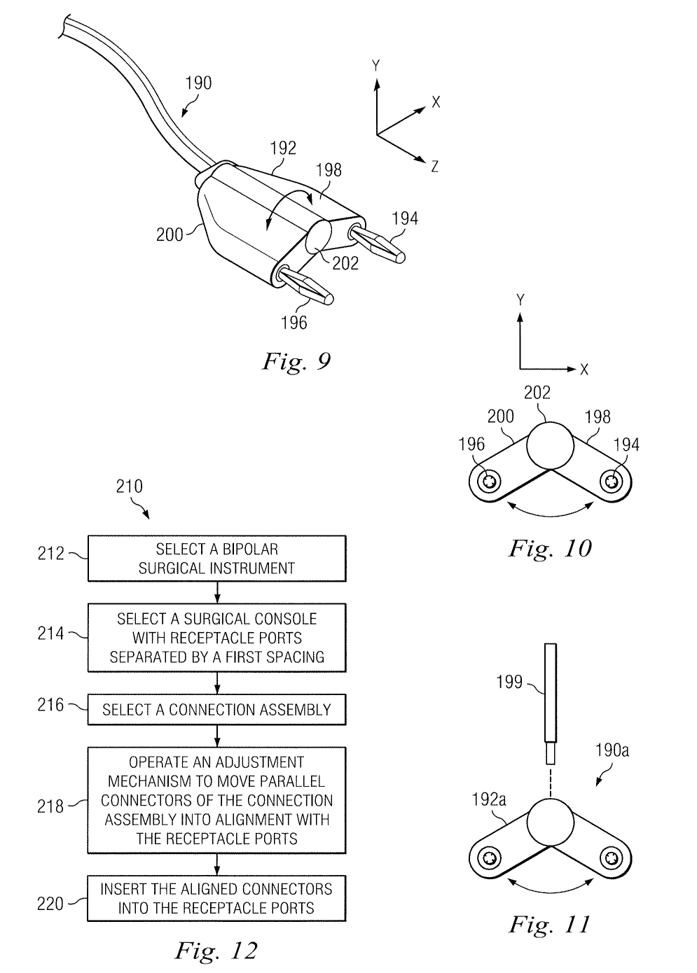

FIG. 9 illustrates a connector assembly 190 including a connector body 192 and a pair of connectors 194, 196. The connector body 192 has portions 198, 200 held in movable connection with each other by a joint or pivot 202. Connector 194 is fixedly connected to portion 198, and connector 196 is fixedly connected to portion 200. The joint 202 may be a hinge, a tether, a section of flexible material, or any other component that permits the portions 198, 200 to pivot relative to one another. The joint may include a spring to bias the portions 198, 200 to extend linearly along the X axis until a user squeezes the portions together to fit the connectors 194, 196 into console receptacle ports. Alternatively, the joint may be freely movable or positively positionable such as by a friction hold. As shown in FIG. 10, moving the connector body portions 198, 200 allows the connectors 194, 196 to move together or apart along the X axis to adjust the spacing between the connectors while maintaining parallel alignment. This adjustability may allow the connectors 194, 196 to fit a variety of consoles having different receptacle port spacings.

FIG. 11, depicts an alternative embodiment of a connector assembly 190a. In this embodiment, the connector assembly 190a includes a connector body 192a and an adjustment mechanism which includes a tool 199. The tool 199 may be, for example, a screwdriver or pliers capable of mating with, attaching to, grabbing, or otherwise connecting with a tool engagement mechanism connected to the connector body 192a. Moving the tool engagement mechanism may activate an internal movement assembly to control movement of the connectors either together or apart, in parallel alignment, along the X axis to adjust the spacing between the connectors. This adjustability may allow the connectors to fit a variety of consoles having different receptacle port spacings.

Any of the previously described connector assemblies may be used by a surgeon or other medical staff to electrically connect a surgical instrument, such as a bipolar surgical instrument, to an electrical console. FIG. 12 is one example of a method 210 for using one of the connector assemblies, such as the connector assembly 190. At step 212, a bipolar surgical instrument may be selected. At step 214, a surgical console may be selected to power the bipolar surgical instrument. The surgical console has a pair of receptacle ports separated by a spacing. At step 216, the connector assembly 190 may be selected to connect the bipolar surgical instrument to the console. At step 218, the portions 198, 200 may be pivoted with respect to each other about the joint 202 so that the parallel connectors 194, 196 are separated by a spacing approximately the same as the spacing between the receptacle ports on the surgical console. After the connectors 194, 196 have been correctly aligned with the receptacle ports, the connectors can be inserted into the receptacle ports to interconnect the bipolar surgical instrument and the surgical console. If a user desires to connect the same bipolar instrument to a different console having a different spacing between receptacle ports, the portions 198, 200 can be pivotally reoriented with respect to each other about the joint 202 so that the parallel connectors 194, 196 are aligned for insertion into the second console. It will be understood that the method of aligning the parallel connectors to the receptacle ports may be altered based upon the adjustment mechanism used to move the parallel connectors.

The connector assemblies of this disclosure may be configured for use with a variety of surgical systems. Example surgical systems in which embodiments of the present invention may be used include ophthalmological systems such as the Constellation® Vision System, the Infiniti® Vision System, the Accurus® Surgical System, and the Laureate® World Phaco System available from Alcon, Inc. with U.S. operations based in Ft. Worth, Tex. While embodiments in this disclosure may be discussed with reference to bipolar surgical consoles, it will be apparent that the invention may be used in any application in which multiple connectors are desirably connected to a console in a controlled manner.

Although several selected embodiments have been illustrated and described in detail, it will be understood that they are exemplary, and that a variety of substitutions and alterations are possible without departing from the spirit and scope of the present invention, as defined by the following claims.

We claim:

1. An electrical connector assembly comprising:
a first elongated connector in parallel alignment with a second elongated connector; and
an adjustment mechanism operable to control movement of the first elongated connector relative to the second elongated connector while maintaining parallel alignment between the elongated connectors.

2. The electrical connector assembly of claim 1 further comprising a connector body having a first slot through which the first elongated connector connects to the connector body.

3. The electrical connector assembly of claim 2 further comprising a second slot through which the second elongated connector connects to the connector body.

4. The electrical connector assembly of claim 1 wherein the adjustment mechanism includes a spring to control the movement of the first elongated connector relative to the second elongated connector while maintaining parallel alignment between the elongated connectors.

5. The electrical connector assembly of claim 1 wherein the adjustment mechanism includes a gear mechanism to control the movement of the first elongated connector relative to the second elongated connector while maintaining parallel alignment between the elongated connectors.

6. The electrical connector assembly of claim 1 further comprising a connector body, wherein the adjustment mechanism includes a tool engagement mechanism attached to the connector body and sized and shaped to receive a tool for controlling the movement of the first elongated connector relative to the second elongated connector while maintaining parallel alignment between the elongated connectors.

7. The electrical connector assembly of claim 1 further comprising a connector body including first portion fixedly connected to the first elongated connector and a second portion fixedly connected to the second elongated connector, wherein the adjustment mechanism movably connects the first and second portions.

8. The electrical connector assembly of claim 7 wherein the adjustment mechanism includes a pivot joint.

9. The electrical connector assembly of claim 7 wherein the adjustment mechanism includes a spring.

10. The electrical connector assembly of claim 1 wherein the first and second elongated connectors are separated by a distance adjustable between 0.50 and 1.50 inches.

11. An electrosurgical system comprising:
a bipolar surgical instrument; and
an electrical connector assembly configured to connect the bipolar surgical instrument to one or more surgical consoles, wherein the electrical connector assembly includes
a first elongated connector in parallel alignment with a second elongated connector; and
an adjustment mechanism operable to control movement of the first elongated connector relative to the second elongated connector while maintaining parallel alignment between the elongated connectors.

12. The electrosurgical system of claim 11 wherein the bipolar surgical instrument is detachably connected to the electrical connector assembly by a coupling.

13. The electrosurgical system of claim 11 wherein the bipolar surgical instrument is an ophthalmological surgical instrument.

14. The electrosurgical system of claim 11 wherein the adjustment mechanism comprises a spring.

15. The electrosurgical system of claim 11 wherein the adjustment mechanism comprises a pivot joint.

16. The electrosurgical system of claim 11 wherein the electrical connector assembly further includes a connector body having a first slot through which the first elongated connector is movably connected to the connector body.

17. The electrosurgical system of claim 11 wherein the electrical connector assembly further includes a connector body and wherein the first elongated connector is fixed relative to the connector body and the second elongated connector is movable relative to the connector body.

18. The electrosurgical system of claim 11 wherein the electrical connector assembly further includes a connector body and wherein both the first and second elongated connectors are movable relative to the connector body.

19. A method comprising:
selecting a bipolar surgical instrument;
selecting a first surgical console adapted for electrical connection to the bipolar surgical instrument, the first surgical console including a first set of paired receptacle ports separated by a first spacing;
selecting a connection assembly, including a first elongated connector in parallel alignment with a second elongated connector and an adjustment mechanism, to interconnect the bipolar surgical instrument and the first surgical console;
operating the adjustment mechanism to move the first elongated connector relative to the second elongated connector while maintaining parallel alignment between the elongated connectors until each of the elongated connectors is in alignment with one of the receptacle ports in the first set; and
inserting each elongated connector into one of the receptacle ports of the first set to connect the bipolar surgical instrument to the first surgical console.

20. The method of claim 19 further comprising coupling the bipolar surgical instrument to the connection assembly.

21. The method of claim 19 further comprising:
removing the elongated connectors from the receptacle ports of the surgical console;
selecting a second surgical console adapted for electrical connection to the bipolar surgical instrument, the second surgical console including a second set of paired receptacle ports separated by a second spacing different than the first spacing;
operating the adjustment mechanism to move the first elongated connector relative to the second elongated connector while maintaining parallel alignment between the elongated connectors until each of the elongated connectors is in alignment with one of the receptacle ports in the second set; and
inserting each elongated connector into one of the receptacle ports of the second set to connect the bipolar surgical instrument to the second surgical console.

22. The method of claim 19 wherein operating the adjustment mechanism includes compressing a spring to move the first elongated connector toward the second elongated connector while maintaining parallel alignment between the elongated connectors.

23. The method of claim 19 wherein operating the adjustment mechanism includes moving at least one of the elongated connectors within a slot.

24. The method of claim 19 wherein operating the adjustment mechanism includes moving a pivot joint to move the first elongated connector toward the second elongated connector while maintaining parallel alignment between the elongated connectors.

25. The method of claim 19 further comprising selecting an adjustment tool and wherein operating the adjustment mechanism includes moving a tool engagement structure with the adjustment tool to move the first elongated connector toward the second elongated connector while maintaining parallel alignment between the elongated connectors.
